(19) United States
(12) Patent Application Publication
(10) Pub. No.: US 2008/0102175 A1
(43) Pub. Date: May 1, 2008

(54) Cooking apparatus and method of displaying caloric information

(75) Inventors: Ki Suk Jeon, Seongnam-si (KR); Kobayashi Shozo, Suwon-si (KR); Jong Chull Shon, Suwon-si (KR); Hyang Ki Kim, Suwon-si (KR)

Correspondence Address: STAAS & HALSEY LLP, SUITE 700, 1201 NEW YORK AVENUE, N.W., WASHINGTON, DC, 20005, US

(73) Assignee: Samsung Electronics Co., Ltd., Suwon-si (KR)

(21) Appl. No.: 11/882,102

(22) Filed: Jul. 30, 2007

(30) Foreign Application Priority Data
Oct. 27, 2006 (KR) 10-2006-0105133

(51) Int. Cl. A23L 1/015; A47J 27/00; A47J 36/00; G01N 33/03

(52) U.S. Cl. 426/233; 99/325; 99/342

(57) ABSTRACT

A cooking apparatus and method of displaying caloric information, capable of calculating fat removed by cooking in terms of caloric value, and displaying the removed caloric value information to a user. The cooking apparatus includes a main body having a cooking chamber and a display unit, a dripping tray placed in the cooking chamber to collects fat discharged from the food during a cooking operation of the cooking apparatus, at least one weight sensor detecting the weight of the dripping tray, and a controller calculating the weight of fat collected in the dripping tray, the weight of which is detected by the weight sensor for the dripping tray, in terms of caloric value, and displaying the calculated result on the display unit.

### CROSS-REFERENCE TO RELATED APPLICATIONS

This application claims the benefit of Korean Patent Application No. 10-2006-0105133, filed on Oct. 27, 2006, in the Korean Intellectual Property Office, the disclosure of which is incorporated herein by reference.

### BACKGROUND OF THE INVENTION

1. Field of the Invention

The present invention relates to a cooking apparatus and method of displaying caloric information. More particularly, a cooking apparatus and method of displaying caloric information, capable of calculating fat removed by cooking in terms of caloric value to display information on a removed caloric value to a user, and displaying caloric information of food after cooking.

2. Description of the Related Art

Food is cooked, for instance, by inserting the food into a gas oven or an electrical oven and directly applying heat to the food, by heating a stew container in which the food is contained, or by inserting the food into a microwave oven and exposing the food to high-frequency radio waves. In addition to the gas oven, the electrical oven, and the microwave oven, a conventional cooking appliance such as a steam oven that cooks the food in a cooking chamber with steam has recently come into the market. Thus, when the food is cooked with steam, the steam is uniformly transmitted to the entire surface of the food, and therefore, the food can be cooked while preserving proper moisture without being burned or undercooked, so that the food is delicious and healthy.

Recently, with regard to cooking the food, the consumers attach more importance to flavor and nutrition of the cooked food rather than cooking method or speed, and pay more attention to various kinds of health information on the food. In particular, a person who wants to adjust his/her weight attempts to track the caloric value of the food ingested, and then ingest the food having a proper caloric value. In this case, the consumers must find and remember information on the caloric value per unit weight of the food ingested, and thus, know the weight of the food ingested.

In order to remove this inconvenience, a cooking appliance displaying caloric information is proposed in Korean Patent Application Publication No. 1992-3804. In this publication, the cooking appliance includes a device of designating type and weight of food by means of automatic cooking. Therefore, information on a caloric value per unit weight of the food, which is stored in a microcomputer, is read out, and thus, the caloric information of the food is obtained. The obtained caloric information is displayed to the consumer.

However, when the food, particularly meat or fish, is cooked, part of oil (fat) is generally removed. The conventional cooking appliance provides only the caloric information of the food before cooking, and thus fails to exactly provide the caloric information of the food after cooking. Further, the conventional cooking appliance does not provide any caloric information on the fat removed in the cooking process. The amount of the fat removed from the food is varied depending on the type of cooking appliance, cooking heat source, and cooking process. As such, when the caloric information on the fat removed in the cooking process is provided to the consumer, the consumer can select the cooking heat source or the cooking process which is more healthful.

### SUMMARY OF THE INVENTION

Accordingly, it is an aspect of the present invention to provide a cooking apparatus and method of displaying caloric information, capable of displaying information on a caloric value of fat removed during a cooking operation, to a consumer.

It is another aspect of the present invention to provide a cooking apparatus and method of displaying caloric information, capable of providing a consumer with accurate information on the caloric value contained in various types of food after each type of food is cooked.

Additional aspects and/or advantages of the invention will be set forth in part in the description which follows and, in part, will be apparent from the description, or may be learned by practice of the invention.

The foregoing and/or other aspects of the present invention are achieved by providing a cooking apparatus which includes a main body having a cooking chamber and a display unit displaying information thereon, a dripping tray placed in the cooking chamber collecting fat discharged from the food during a cooking operation of the cooking apparatus, at least one weight sensor detecting a weight of the dripping tray, and a controller calculating the weight of fat collected in the dripping tray, the weight of which is detected by the weight sensor for the dripping tray, in terms of caloric value, and displaying the calculated result on the display unit.

According to an aspect of the present invention, the weight sensor for the dripping tray is installed under the dripping tray, and detects the weight of the fat collected in the dripping tray. Further, the weight sensor for the dripping tray is set a zero point when the dripping tray is placed thereon.

Further, according to an aspect of the present invention, the controller controls the weight sensor for the dripping tray to detect the weight of the fat in the dripping tray when a caloric information display order is input by a user, and displays the caloric value corresponding to the detected weight of the fat.

It is another aspect of the present invention to provide a method of displaying caloric information, in which a caloric value of fat removed from food is displayed. The method includes cooking the food, detecting a weight of the fat removed during cooking, calculating the detected weight of the fat in terms of caloric value, and displaying the caloric value corresponding to the weight of the removed fat. According to an aspect of the present invention, detecting a weight of the fat removed in the cooking step includes detecting a weight of a dripping tray that is installed under the food and collects the fat discharged from the food. Further, according to an aspect of the present invention, the caloric value corresponding to the weight of the removed fat is displayed when a caloric information display order is input by a consumer or automatically displayed when the cooking is completed.

It is another aspect of the present invention to provide a cooking apparatus, which includes a main body having a cooking chamber and a display unit displaying information thereon, a dripping tray placed in the cooking chamber collecting fat discharged from the food during a cooking operation of the cooking apparatus, at least one first weight sensor detecting a weight of the dripping tray, at least one second weight sensor detecting a weight of food, and a controller subtracting a caloric value based on a weight of fat collected in the dripping tray from a caloric value based on an initial weight of the food, and displaying the subtracted result after cooking the food on the display unit.

According to an aspect of the present invention, the food includes at least one type that is selected from a cooking menu set in the cooking apparatus by means of a user, or is input through a bar code reader or a radio frequency identification (RFID) reader that reads out the type of the food.

According to an aspect of the present invention, the cooking apparatus further includes a shelf on which the food is placed. According to an aspect of the present invention, the second weight sensor for the food is installed under the shelf, and detects the initial weight of the food.

Further, according to an aspect of the present invention, the controller controls the second weight sensor for the food to detect the initial weight of the food before cooking to thereby obtain a caloric value of the food before cooking, controls the first weight sensor for the dripping tray to detect the weight of the fat in the dripping tray, when a caloric information display order is input by a consumer, to thereby obtain a caloric value corresponding to the weight of the removed fat, subtracts the caloric value corresponding to the weight of the removed fat from the caloric value of the food before cooking, and displays a current caloric value of the food.

According to an aspect of the present invention, the cooking apparatus further includes a memory storing information on the caloric value per unit weight of each type of food. According to an aspect of the present invention, the controller obtains the caloric value of the food before cooking based upon the information stored in the memory, on the caloric value per unit weight of each type of food and based upon input information on the type of food. Further, according to an aspect of the present invention, the controller reads out information on a type of the food and the caloric value per unit weight of the food from a bar code or radio frequency identification (RFID), and obtains the caloric value of the food before cooking.

Furthermore, according to an aspect of the present invention, the controller displays any one of the caloric value of the food before cooking, the caloric value of the food after cooking, and a removed caloric value by means of an order of a user or automatically

It is yet another aspect of the invention to provide a method of displaying caloric information, in which a caloric value of food is displayed. The method includes detecting an initial weight of the food to obtain the caloric value of the food before cooking, cooking the food, detecting a weight of the fat removed during cooking to obtain removed caloric value, subtracting the removed caloric value from the caloric value of the food before cooking to obtain the caloric value of the food after cooking, and displaying the caloric value of the food after cooking.

According to an aspect of the present invention, obtaining the caloric value of the food before cooking includes obtaining the caloric value of the food before cooking based upon the information on the type of food, on the initial weight of the food, and on the caloric value per unit weight of each type of food.

Further, according to an aspect of the present invention, the type of food is selected from a cooking menu or be read out from a bar code or radio frequency identification (RFID).

Also, according to an aspect of the present invention, the information on the caloric value per unit weight of each type of food may be stored in a memory of a cooking apparatus or be read out from a bar code or a radio frequency identification (RFID) of each type of food.

Meanwhile, according to an aspect of the present invention, obtaining the caloric value removed by the cooking includes detecting a weight of a dripping tray that is installed below the food and collects the fat discharged from the food, and calculating the caloric value corresponding to the detected weight of the fat.

Further, according to an aspect of the present invention, the caloric value of the food after cooking is displayed when a caloric information display order is input by a consumer or be automatically displayed when the cooking is completed. Also, according to an aspect of the present invention, the method further includes displaying the caloric value of the food before cooking or a removed caloric value.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a perspective view illustrating a cooking apparatus according to a first embodiment of the present invention;

FIG. 2 is a sectional view illustrating a cooking apparatus according to a first embodiment of the present invention;

FIG. 3 is a block diagram illustrating the construction of a cooking apparatus according to a first embodiment of the present invention;

FIG. 4 is a flowchart illustrating the control operation of a cooking apparatus according to a first embodiment of the present invention;

FIG. 5 is a perspective view illustrating a cooking apparatus according to a second embodiment of the present invention;

FIG. 6 is a block diagram illustrating the construction of a cooking apparatus according to a second embodiment of the present invention; and

FIG. 7 is a flowchart illustrating the control operation of a cooking apparatus according to a second embodiment of the present invention.

### DETAILED DESCRIPTION OF THE PREFERRED EMBODIMENTS

Reference will now be made in detail to the embodiments of the present invention, examples of which are illustrated in the accompanying drawings, wherein like reference numerals refer to the like elements throughout. The embodiments are described below to explain the present invention by referring to the figures.

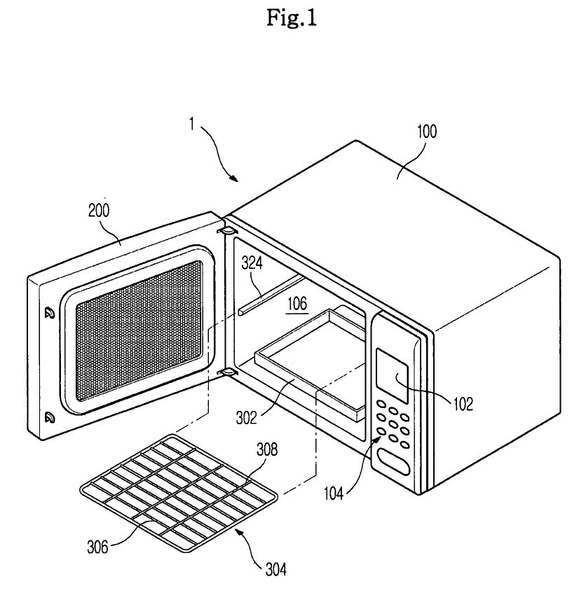

FIG. 1 is a perspective view illustrating a cooking apparatus according to a first embodiment of the present invention. As shown in FIG. 1, the cooking apparatus 1 according to an embodiment of the present invention comprises a main body 100 forming an external appearance. The main body 100 comprises a cooking chamber 106 on one side thereof, in which food is placed and cooked. The main body 100 further comprises an input unit 104 on the other side thereof, controlling operation of the cooking apparatus, and a control panel having a display unit 102 informing an operated state. The main body 100 also comprises a door 200 opening and closing the cooking chamber 106 at the front of the cooking chamber 106. In the cooking apparatus 1 according to an embodiment of the present invention, the input unit 104 is additionally provided including a caloric information display button of ordering display of caloric information. The cooking apparatus 1 is not limited to this structure. Accordingly, the structure of the cooking apparatus 1 may vary, as necessary. For example, it does not matter that cooking apparatus 1 is constructed in such a manner that the control panel is located on an upper side, that the cooking chamber is formed below the control panel, and that the door is open from top to bottom.

The cooking chamber 106 further comprises shelf struts 324 on opposite sidewalls thereof, which support a shelf 304 on which the food is placed. The cooking chamber 106 comprises a dripping tray 302, which collects moisture or fat dripping down from the food placed on the shelf 304 in the cooking process. The shelf 304 is constructed in such a manner that transverse ribs 306 intersect with longitudinal ribs 308. The structure of the shelf 304 may vary as long as moisture or fat falling out of the food placed on the shelf 304 can pass through the shelf 304. Further, the dripping tray 302 can be constructed in various structures.

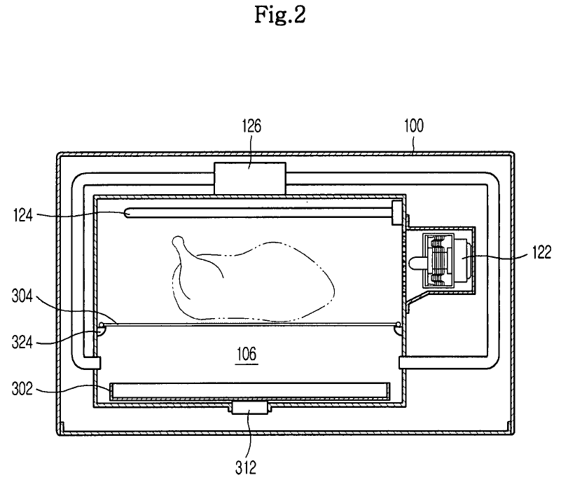

FIG. 2 is a sectional view illustrating a cooking apparatus according to a first embodiment of the present invention. As shown in FIG. 2, a weight sensor 312 is installed below the dripping tray 302, to detect the weight of the dripping tray 302. The weight sensor 312 for the dripping tray is constructed to set a zero point when the dripping tray 302 is placed thereon, so that a weight of the fat collected in the dripping tray 302 can be accurately detected. Although only one weight sensor 312 is installed below the dripping tray 302 as illustrated in FIG. 2, according to another embodiment of the present invention, four weight sensors are installed at four places below the dripping tray respectively, and the average is taken of their values. In addition, the present invention is not limited to any particular type or number of sensors, and may vary as necessary, as long as the weight of the dripping tray 302 can be detected.

The cooking chamber 106 further comprises a high-frequency heater 122 heated by a magnetron on one side thereof. The cooking chamber 106 further comprises a radiant heater 124 such as a grill heater on an upper portion thereof. Further, a steam heater 126 that heats water to form steam and supplies the formed steam into the cooking chamber 106 is installed between the main body 100 and the cooking chamber 106. In addition, various heat sources capable of cooking the food in the cooking chamber may be installed as needed. Among the above-described heaters, some may be installed.

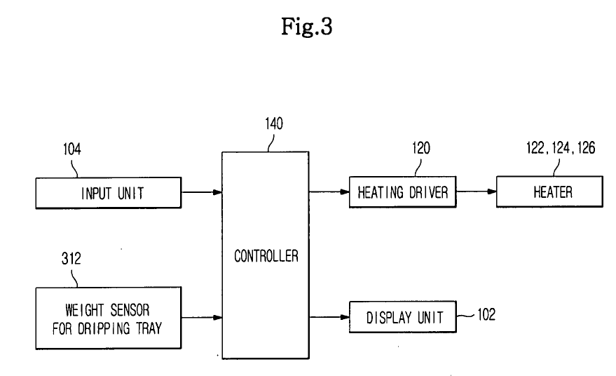

The construction of the cooking apparatus according to a first embodiment of the present invention will be functionally described with reference to FIG. 3. A user inputs contents to be cooked such as cooking item, cooking temperature, cooking time, etc. through the input unit 104. The input contents are sent to a controller 140. The controller 140 drives a heating driver 120 to operate the respective heaters 122, 124 and 126 according to the cooking conditions input by the user. Further, the controller 140 displays the cooking conditions of the user or an operated state of the cooking apparatus 1 on the display unit 102, thereby providing such information to the user. Further, when the caloric information display order of the user is input through the input unit 104 or when the cooking is completed, the controller 140 orders operation of the weight sensor 312 for the dripping tray, detects the weight of the fat collected in the dripping tray 302, calculates the detected weight of the fat in terms of caloric value, and displays the calculated caloric value. Typically, the caloric value of the fat is calculated by multiplying by approximately 9 kcal/g, irrespective of the type of food from which the fat is extracted.

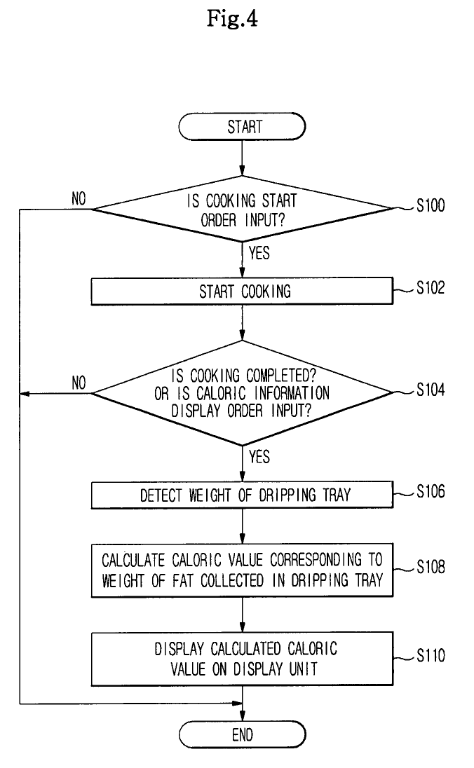

The control operation of the cooking apparatus according to a first embodiment of the present invention will be described with reference to FIG. 4. As shown in FIG. 4, in operation 100 it is determined whether the user places food on the shelf 304 in the cooking chamber 106 in order to cook the food and then inputs a cooking start order. From operation 100, the process moves to operation 102, where when the cooking start order is input, the controller 140 operates at least one of the heaters 122, 124 and 126 according to cooking conditions, thereby cooking the food. When the food begins to be cooked, moisture, fat, etc. are removed from the food placed on the shelf 304, and then are collected into the dripping tray 302. At this time, because the moisture is easily evaporated in the cooking chamber, only the fat is actually left in the dripping tray 302.

Next, from operation 102, the process move to operation 104, where it is determined whether a caloric information display order is input by the user during or after cooking. Alternatively, when the information on the removed caloric value is adapted to be automatically displayed to the user when the cooking is completed, it is determined whether the cooking is completed. Therefore, the caloric information can be manually displayed only when the user's display order is input, or automatically displayed when the cooking is completed. In this manner, the present invention includes both automatically displaying the caloric information when the cooking is completed and manually displaying the caloric information only when the user's display order is input, for example.

When it is determined that the cooking is completed, or when the caloric information display order of the user is input in operation 104, the process moves to operation 106, where the controller 140 controls the weight sensor 312 for the dripping tray to detect a weight of the fat collected in the dripping tray 302. Thus, when receiving a detection order signal from the controller 140, the weight sensor 312 for the dripping tray is operable and then detects the weight of the fat. Alternatively, while continuing to detect the weight of the fat, when receiving a detection order signal from the controller 140, the weight sensor 312 for the dripping tray may send the weight of the fat detected at that time to the controller 140.

Next, from operation 106, the process moves to operation 108, where the controller 140 calculates a caloric value corresponding to the weight of the removed fat which is detected by the weight sensor 312 for the dripping tray. As described above, the caloric value of the fat can be calculated by multiplying by approximately 9 kcal/g regardless of the type of food from which the fat is extracted. Subsequently, the process then moves to operation 110, where the calculated caloric value of the fat is displayed on the display unit 102, so that the user is informed of how much the caloric value of the fat is removed during cooking.

Thus, according to the first embodiment of the present invention, the cooking apparatus informs the consumer, who is interested in the caloric value of the food for the purpose of medical treatment or weight control, of the caloric value of the removed fat with accuracy. Furthermore, the consumer can directly compare and verify the caloric value of the fat that is removed from the food and is varied depending on a type of cooking appliance, a type of cooking heat source, and a cooking process, so that he/she can be provided with the information on the cooking heat source or the cooking process which is more healthful.

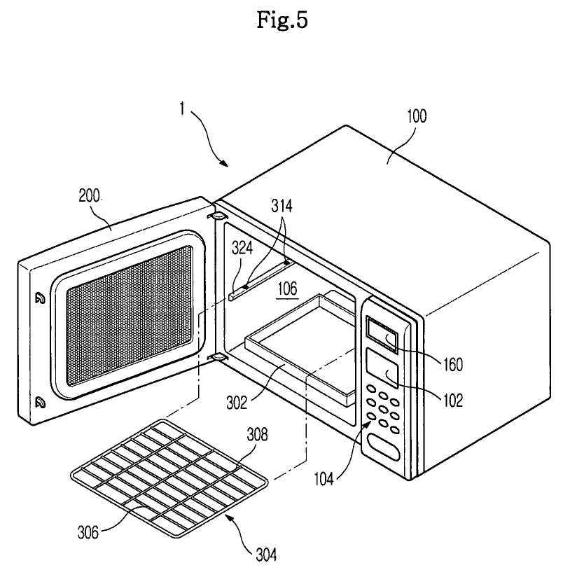

Hereinafter, a cooking apparatus according to a second embodiment of the present invention and a method of displaying caloric information will be described with reference to FIGS. 5, 6 and 7. The same parts as in the first embodiment are assigned the same reference numerals, and thus a description thereof will be omitted.

FIG. 5 is a perspective view illustrating a cooking apparatus according to a second embodiment of the present invention. The cooking apparatus 1 further comprises a bar code reader 160, and weight sensors 314, for food, mounted on shelf struts 324 so as to be able to detect the weight of the food placed on a shelf 304, in addition to the construction of the cooking apparatus according to the first embodiment.

The bar code reader 160 is an input device to read out information from a bar code that is attached to the container of the food and contains the manufacturer details and type of the food as well as other pieces of cooking information. Although the bar code reader 160 illustrated in FIG. 5 is an internal type, it may be implemented as an external type. It does not matter that the bar code reader 160 is replaced with another similar information input device such as a radio frequency identification (RFID) reader. The weight sensors 314 for the food are installed two in number on each shelf strut 324, and detects the weight of the shelf 304 placed thereon. In this case, preferably, an average is taken of values of the weight sensors 314. Like the weight sensor 312 for the dripping tray, the weight sensors 314 for the food are constructed to set a zero point when the shelf 304 is placed thereon, so that only the weight of the food can be accurately detected. Although the weight sensors 314 for the food are constructed to be installed on the shelf struts 324, the present invention is not limited thereto and may vary so long as the weight of the food can be detected. For example, a measuring tool such as a measuring tray may be separately installed on an external upper side of the cooking apparatus, and thereby the weight of the food may be first measured before inputting it into the cooking chamber 106.

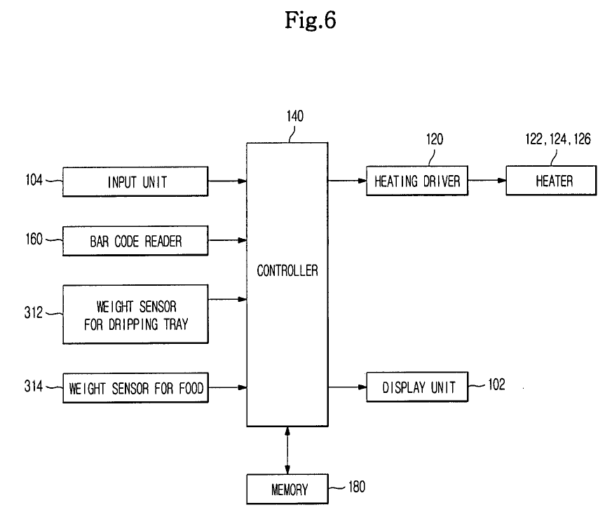

The construction of the cooking apparatus according to a second embodiment of the present invention will be functionally described with reference to FIG. 6. Here, the same parts as in the first embodiment will be not described. The cooking apparatus 1 according to a second embodiment comprises the bar code reader 160, the weight sensors 314 for the food, and a memory 180. The bar code reader 160 reads out information from a bar code attached to the food to be cooked, and input a type of the food into the cooking apparatus, as described above. It does not matter that the bar code reader 160 is replaced with the input device such as the RFID reader. The memory 180 stores various pieces of cooking information which a regular cooking apparatus has, for instance, a type of each food based on an automatic cooking menu, and optimal cooking conditions of the type of corresponding food. Thus, the consumer can directly select and input the type of food to be cooked from the cooking menu stored in the memory 180 mounted in the main body of the cooking apparatus.

Herein, the type of food can be directly selected and input from the cooking menu by the consumer, or read out from the bar code. However, the type of food is selected and input from the automatic cooking menu stored in the memory 180 without the bar code reader. Further, the type of food may be input through the bar code reader or the RFID reader without the automatic cooking menu.

Herein, the memory 180 stores information on the caloric value per weight of each food in addition to the type of food based on the automatic cooking menu and the optimal cooking conditions, as follows. The following data is information on the standard caloric value of each food used in the Korean Dietetic Association.

TABLE 1
Information on Caloric Value per Unit Weight of Food

| | | | Unit Weight | Caloric Value |
|---|---|---|---|---|
| | Food | | (g) | (kcal) |
| Meat | Poultry | Meat with Skin | 40 | 100 |
| | meat | Lean Meat without | 40 | 50 |
| | | Skin and Fat | | |
| | Pork | Tenderloin | 40 | 75 |
| | | Defatted Lean | 40 | 50 |
| | | Meat | | |
| | Beef | Shank, Fore Rump | 40 | 50 |
| | | Sirloin, Tenderloin | 40 | 75 |
| | | Ribs | 50 | 100 |
| | . . . | . . . | | |
| Fish | Salmon | | 50 | 50 |
| | Mackerel | | 50 | 75 |
| | Saury | | 50 | 75 |
| | Eel | | 50 | 100 |
| | . . . | | | |
| . . . | . . . | | | |

Herein, the case in which the information on the caloric value per unit weight of each food is stored in the memory 180 has been described. However, if the bar code has the information on the type of each food and the information on the caloric value per unit weight of the corresponding food, it does not matter that the information on the caloric value per unit weight of each food is provided to the controller 140 through the bar code reader 160.

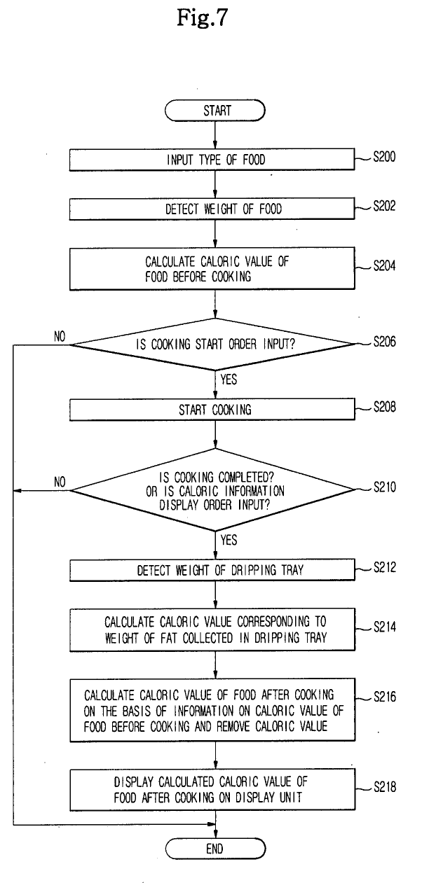

The control operation of the cooking apparatus according to a second embodiment of the present invention will be described with reference to FIG. 7. As shown in FIG. 7, a user places a food on the shelf 304 in the cooking chamber 106 in order to cook the food, and then closes the door 200. Then, in operation 200, the user selects a type of food from an automatic cooking menu, and inputs the type of food. As described above, in the case of inputting the type of food through the bar code, the type of food is first input through the bar code reader 160, and then the food is placed on the shelf 304 in the cooking chamber 106. When the food is placed on the shelf 304 in the cooking chamber 106, the process then moves to operation 202 where the controller detects the weight of the food by means of the weight sensors 314 for the food. When the type of food is input in operation 200, and when the weight of the food to be cooked is detected in operation 202, the process then moves to operation 204 where the controller 140 calculates a caloric value of the food before the cooking by means of the information, stored in the memory 180, on the caloric value per unit weight of the corresponding type of food. It is not indispensably necessary to perform this calculating process at present, and thus, the calculating process can be performed when the caloric information is displayed in the future.

Next, in operation 206, it is determined whether a cooking start order is input. When the cooking start order is input, the process moves to operation 208 where the controller 140 operates at least one of the heaters 122, 124 and 126 according to cooking conditions, thereby cooking the food. When the food begins to be cooked, moisture, fat, etc. are removed from the food placed on the shelf 304, and are then collected into the dripping tray 302. Next, in operation 210, it is determined whether a caloric information display order is input by the user during or after cooking.

Alternatively, when the information on the caloric value of the removed fat is adapted to be automatically displayed to the user when the cooking is completed, it is determined whether the cooking is completed in operation 210.

Subsequently, when it is determined that the cooking is completed in operation 210, or when the caloric information display order of the user is input, the process moves to operation 212 where the controller 140 controls the weight sensor 312 for the dripping tray to detect a weight of the fat collected in the dripping tray 302. When receiving a detection order signal from the controller 140, the weight sensor 312 for the dripping tray can be operated and then detect the weight of the fat. Alternatively, while continuing to detect the weight of the fat, when receiving a detection order signal from the controller 140, the weight sensor 312 for the dripping tray sends the weight of the fat detected at that time to the controller 140.

Next, the process moves to operation 214 where the controller 140 calculates the caloric value corresponding to the weight of the removed fat which is detected by the weight sensor 312 for the dripping tray. As described above, according to an embodiment of the present invention, the caloric value of the fat is calculated by multiplying by approximately 9 kcal/g regardless of the type of food from which the fat is extracted. Then, the process moves to operation 216, where the controller 140 calculates the caloric value of the food after cooking from both the caloric value, calculated in operation 204, of the food before cooking and the caloric value of the removed fat obtained in operation 214. The process then moves to operation 218, where the calculated caloric value, calculated in operation 216, of the food after cooking is displayed on the display unit 102, so that the user is informed of how much the caloric value of the food has changed. In operation 218 of displaying the caloric information, according to an embodiment of the present invention, the display unit 102 alternately and automatically displays information on the caloric value of the removed fat and initial information on the caloric value of the food before cooking in addition to the information on the caloric value of the food after cooking, or displays each piece of caloric information in a toggle way when the user repetitively pushes a caloric information display order button.

Thus, according to the second embodiment of the present invention, the cooking apparatus can accurately provide the information on the caloric value that the food ingested finally by the consumer has.

As described above, according to the first embodiment of the present invention, the caloric information of the fat removed in the cooking process of the cooking apparatus can be displayed to the consumer. Further, the consumer can directly compare the caloric values of the fat that is removed from the food depending on the type of cooking appliance, cooking heat source, and cooking process, so that he/she can be provided with the information on the cooking heat source or the cooking process which is healthiest.

In addition, according to the second embodiment of the present invention, the caloric information of the food after cooking and that the consumer actually takes rather than that of the food before cooking can be accurately provided.

Although few embodiments of the present invention have been shown and described, it would be appreciated by those skilled in the art that changes may by made in these embodiments without departing from the principles and spirit of the invention, the scope of which is defined in the claims and their equivalents.

What is claimed is:

1. A cooking apparatus comprising:
a main body having a cooking chamber and a display unit to display information thereon;
a dripping tray placed in the cooking chamber to collect fat discharged from food during a cooking operation of the cooking apparatus;
at least one weight sensor detecting a weight of the dripping tray; and
a controller calculating a weight of the fat collected in the dripping tray, the weight of which is detected by the weight sensor for the dripping tray, in terms of caloric value, and displaying the calculated result on the display unit.

2. The cooking apparatus as claimed in claim 1, wherein the weight sensor for the dripping tray is installed under the dripping tray, and detects the weight of the fat collected in the dripping tray.

3. The cooking apparatus as claimed in claim 2, wherein the weight sensor for the dripping tray sets an initial zero point when the dripping tray is placed thereon.

4. The cooking apparatus as claimed in claim 1, wherein the controller controls the weight sensor for the dripping tray to detect the weight of the fat in the dripping tray, when a caloric information display order is input by a user, and displays the caloric value corresponding to the detected weight of the fat.

5. The cooking apparatus as claimed in claim 1, wherein the controller controls the weight sensor for the dripping tray to detect the weight of the fat in the dripping tray when cooking is completed, and automatically displays the caloric value corresponding to the detected weight of the fat.

6. A method of displaying caloric information, in which a caloric value of fat removed from food is displayed, the method comprising:
cooking the food;
detecting a weight of the fat removed during cooking;
calculating the detected weight of the fat in terms of caloric value; and
displaying the calculated caloric value corresponding to the detected weight of the removed fat.

7. The method as claimed in claim 6, wherein detecting a weight of the fat removed during cooking comprises detecting a weight of a dripping tray which is installed under the food and collects the fat discharged from the food.

8. The method as claimed in claim 6, wherein the caloric value corresponding to the weight of the removed fat is displayed, when a caloric information display order is input by a consumer or is automatically displayed when the cooking is completed.

9. A cooking apparatus comprising:
a main body having a cooking chamber and a display unit to display information thereon;
a dripping tray placed in the cooking chamber to collect fat discharged from food during a cooking operation of the cooking apparatus;
at least one first weight sensor detecting a weight of the dripping tray;
at least one second weight sensor detecting a weight of the food; and
a controller subtracting a caloric value based on a weight of fat collected in the dripping tray from a caloric value, based on an initial weight of the food, and displaying the subtracted result after cooking the food, on the display unit.

10. The cooking apparatus as claimed in claim 9, wherein the food comprises at least one type which is selected from a cooking menu set in the cooking apparatus by a user.

11. The cooking apparatus as claimed in claim 9, further comprising a bar code reader or a radio frequency identification (RFID) reader that reads out the type of the food.

12. The cooking apparatus as claimed in claim 9, further comprising a shelf on which the food is placed,
wherein the second weight sensor for the food is installed under the shelf and detects the initial weight of the food.

13. The cooking apparatus as claimed in claim 9, wherein the controller controls the second weight sensor for the food to detect the initial weight of the food before cooking, to thereby obtain a caloric value of the food before cooking, controls the first weight sensor for the dripping tray to detect the weight of the fat in the dripping tray when a caloric information display order is input by a consumer, to thereby obtain a caloric value corresponding to the weight of the removed fat, subtracts the caloric value corresponding to the weight of the removed fat from the caloric value of the food before cooking, and displays a current caloric value of the food based upon the subtraction result.

14. The cooking apparatus as claimed in claim 9, wherein the controller controls the second weight sensor for the food to detect the initial weight of the food before cooking to thereby obtain a caloric value of the food before cooking, controls the first weight sensor for the dripping tray to detect the weight of the fat in the dripping tray when the food is completely cooked, to thereby obtain a caloric value corresponding to the weight of the removed fat, subtracts the caloric value corresponding to the weight of the removed fat from the caloric value of the food before cooking, and automatically displays a current caloric value of the food based upon the subtraction result.

15. The cooking apparatus as claimed in claim 13 or 14, further comprising a memory storing information on the caloric value per unit weight of each type of food,
wherein the controller obtains the caloric value of the food before cooking on the basis of the information, stored in the memory, on the caloric value per unit weight of each type of food and of input information on the type of food.

16. The cooking apparatus as claimed in claim 13, wherein the controller reads out information on a type of the food and the caloric value per unit weight of the food from a bar code or a radio frequency identification (RFID), and obtains the caloric value of the food before cooking.

17. The cooking apparatus as claimed in claim 13, wherein the controller displays at least one of the caloric value of the food before cooking, the caloric value of the food after cooking, and a removed caloric value based upon an order of a user or automatically.

18. A method of displaying caloric information, in which a caloric value of food is displayed, the method comprising:
detecting an initial weight of the food to obtain the caloric value of the food before cooking;
cooking the food;
detecting a weight of the fat removed during cooking, to obtain removed caloric value;
subtracting the removed caloric value from the caloric value of the food before cooking to obtain the caloric value of the food after cooking; and
displaying the caloric value of the food after cooking based upon the subtraction result.

19. The method as claimed in claim 18, wherein obtaining the caloric value of the food before cooking comprises obtaining the caloric value of the food before cooking based upon information on a type of food, on an initial weight of the food, and on a caloric value per unit weight of each type of food.

20. The method as claimed in claim 19, wherein the type of food is selected from a cooking menu or is read out from a bar code or a radio frequency identification (RFID).

21. The method as claimed in claim 19, wherein the information on the caloric value per unit weight of each type of food is stored in a memory of a cooking apparatus or is read out from a bar code or a radio frequency identification (RFID) of each type of food.

22. The method as claimed in claim 18, wherein obtaining the caloric value removed by the cooking comprises detecting a weight of a dripping tray which is installed below the food, and collects the fat discharged from the food, and calculating the caloric value corresponding to the detected weight of the fat.

23. The method as claimed in claim 18, wherein the caloric value of the food after cooking is displayed when a caloric information display order is input by a consumer or is automatically displayed when the cooking is completed.

24. The method as claimed in claim 18, further comprising displaying the caloric value of the food before cooking or a removed caloric value.
